(19) United States
(12) Patent Application Publication
(10) Pub. No.: US 2025/0336524 A1
(43) Pub. Date: Oct. 30, 2025

(54) METHOD FOR ASSESSING BACTEREMIA AND BACTEREMIA ASSESSING SYSTEM

(71) Applicant: China Medical University, Taichung City (TW)

(72) Inventors: Der-Yang Cho, Taichung City (TW); Po-Ren Hsueh, Taichung City (TW); Hong-Mo Shih, Taichung City (TW); Yu-Hsin Chang, Taichung City (TW); Chiung-Tzu Hsiao, Taichung City (TW); Yu-Chang Chang, Taichung City (TW); Hsin-Yu Lai, Taichung City (TW); Hsiu-Hsien Lin, Taichung City (TW); Chien-Chih Chen, Toufen City (TW); Lin-Chen Hsu, Tainan City (TW); Shih-Yun Wu, Taichung City (TW)

(73) Assignee: China Medical University, Taichung City (TW)

(21) Appl. No.: 18/671,038

(22) Filed: May 22, 2024

(30) Foreign Application Priority Data
Apr. 25, 2024 (TW) 113115481

(51) Int. Cl. G16H 50/20

(52) U.S. Cl. CPC G16H 50/20

(57) ABSTRACT

A method for assessing bacteremia includes the following steps. A blood analysis database is provided. A model establishing step is performed, wherein a plurality of reference cell population data, a plurality of reference complete blood counting data and a plurality of reference white blood cell differential counting data of the blood analysis database are trained to achieve a convergence by a machine learning algorithm model so as to obtain a bacteremia assessing classifier. A blood analysis data of a subject is provided, wherein the blood analysis data includes a cell population data, a complete blood counting data and a white blood cell differential counting data. An assessing step is performed, wherein the blood analysis data is analyzed by the bacteremia assessing classifier so as to obtain an assessing result of bacteremia of the subject.

### RELATED APPLICATIONS

This application claims priority to Taiwan Application Serial Number 113115481, filed Apr. 25, 2024, which is herein incorporated by reference.

### BACKGROUND

### Technical Field

The present disclosure relates to a medical information analysis method and a system thereof. More particularly, the present disclosure relates to a method for assessing bacteremia and a bacteremia assessing system.

### Description of Related Art

Bacteremia is a disease in which viable bacteria are present in the bloodstream. If the bacteremia is not diagnosed promptly and treated with appropriate antibiotics, the patient is at high risk of death.

In the current clinical procedures, a blood culture method is used to diagnose whether a subject has the bacteremia or not. However, it takes an average of 16 hours to 25 hours to obtain a positive test report, and approximately one-third of positive test results are false positive results caused by bacteria that are not present in the blood sample but introduced into the culture bottle during the blood collection process. Hence, subjects need to undergo additional laboratory tests and unnecessary antibiotic treatments, which consumes more time and cost, resulting in a significant increase in medical expenses.

Therefore, how to provide a rapid and accurate method to identify whether a subject has bacteremia or not so as to formulate an appropriate treatment strategy, has become the goal of the relevant academic and industry development.

### SUMMARY

According to one aspect of the present disclosure, a method for assessing bacteremia includes the following steps. A blood analysis database is provided, wherein the blood analysis database includes a plurality of reference cell population data, a plurality of reference complete blood counting data and a plurality of reference white blood cell differential counting data. A model establishing step is performed, wherein the plurality of reference cell population data, the plurality of reference complete blood counting data and the plurality of reference white blood cell differential counting data are trained to achieve a convergence by a machine learning algorithm model so as to obtain a bacteremia assessing classifier. A blood analysis data of a subject is provided, wherein the blood analysis data includes a cell population data, a complete blood counting data and a white blood cell differential counting data. An assessing step is performed, wherein the blood analysis data is analyzed by the bacteremia assessing classifier so as to obtain an assessing result of bacteremia of the subject.

According to another aspect of the present disclosure, a bacteremia assessing system includes a non-transitory machine-readable medium and a processor. The non-transitory machine-readable medium is for storing a blood analysis data of a subject, wherein the blood analysis data includes a cell population data, a complete blood counting data and a white blood cell differential counting data. The processor is signally connected to the non-transitory machine-readable medium, wherein the processor includes a bacteremia assessing classifier, and the blood analysis data is analyzed by the bacteremia assessing classifier so as to obtain an assessing result of bacteremia of the subject.

### BRIEF DESCRIPTION OF THE DRAWINGS

The patent or application file contains at least one drawing executed in color. Copies of this patent or patent application publication with color drawing(s) will be provided by Office upon request and payment of the necessary fee. The present disclosure can be more fully understood by reading the following detailed description of the embodiment, with reference made to the accompanying drawings as follows:

FIG. 1 is a flow chart of a method for assessing bacteremia according to one embodiment of the present disclosure.

FIG. 2 is a block diagram of a bacteremia assessing system according to another embodiment of the present disclosure.

FIG. 3 shows the receiver operating characteristic curves of bacteremia assessing systems of Example 1 to Example 5.

FIG. 4 shows the precision-recall curves of the bacteremia assessing systems of Example 1 to Example 5.

FIG. 5 shows the analysis results of the correlations of different feature marks to the bacteremia assessing system of Example 1 used to assess bacteremia.

FIG. 6 shows the analysis results of the correlations of different feature marks to the bacteremia assessing system of Example 3 used to assess bacteremia.

### DETAILED DESCRIPTION

The present disclosure will be further exemplified by the following specific embodiments. However, the embodiments can be applied to various inventive concepts and can be embodied in various specific ranges. The specific embodiments are only for the purposes of description and are not limited to these practical details thereof.

### [Method for Assessing Bacteremia of the Present Disclosure]

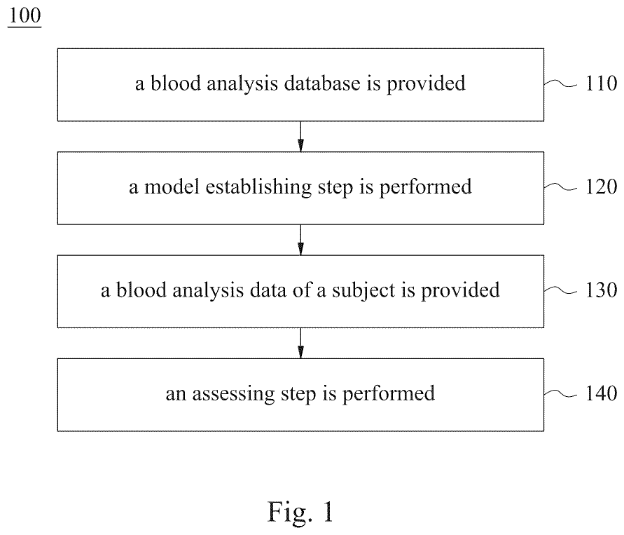

Reference is made to FIG. 1, which is a flow chart of a method 100 for assessing bacteremia according to one embodiment of the present disclosure. The method 100 for assessing bacteremia includes Step 110, Step 120, Step 130 and Step 140.

In Step 110, a blood analysis database is provided, wherein the blood analysis database includes a plurality of reference cell population data, a plurality of reference complete blood counting data and a plurality of reference white blood cell differential counting data. In detail, the blood analysis database can be a dataset obtained from the results of blood measurements in electronic medical records (EMRs) of subjects in a hospital, wherein the reference cell population data, the reference complete blood counting data and the reference white blood cell differential counting data can be obtained by analyzing blood samples of different subjects with a hematology analyzer.

In particular, the reference cell population data can include a reference parameter data set, wherein the reference parameter data set can be obtained by analyzing the blood samples of the different subjects by a volume, conductivity and scatter (VCS) method. Further, in the volume, conductivity and scatter method, a volume of a cell is measured by an electrical impedance method, the internal composition of the cell is simultaneously analyzed by a radio frequency current, and a cell granularity is measured by scattered lights at different angles. Hence, the cell can undergo multi-parameter analysis in its original, unprocessed state, and the detection efficiency and the detection accuracy of the present disclosure can be enhanced.

The reference parameter data set can include a reference cell volume data subset, a reference conductivity data subset, a reference median angle light scatter data subset, a reference upper median angle light scatter data subset, a reference lower median angle light scatter data subset, a reference low angle light scatter data subset and a reference axial light loss data subset, wherein each of the reference cell volume data subset, the reference conductivity data subset, the reference median angle light scatter data subset, the reference upper median angle light scatter data subset, the reference lower median angle light scatter data subset, the reference low angle light scatter data subset and the reference axial light loss data subset can include a mean data and a standard deviation data.

In detail, the reference cell volume data subset can include a reference mean volume of neutrophil data, a reference volume standard deviation of neutrophil data, a reference mean volume of lymphocyte data, a reference volume standard deviation of lymphocyte data, a reference mean volume of monocyte data, a reference volume standard deviation of monocyte data, a reference mean volume of eosinophil data and a reference volume standard deviation of eosinophil data.

Further, the reference conductivity data subset can include a reference mean conductivity of neutrophil data, a reference conductivity standard deviation of neutrophil data, a reference mean conductivity of lymphocyte data, a reference conductivity standard deviation of lymphocyte data, a reference mean conductivity of monocyte data, a reference conductivity standard deviation of monocyte data, a reference mean conductivity of eosinophil data and a reference conductivity standard deviation of eosinophil data.

Further, the reference median angle light scatter data subset can include a reference mean median angle light scatter of neutrophil data, a reference median angle light scatter standard deviation of neutrophil data, a reference mean median angle light scatter of lymphocyte data, a reference median angle light scatter standard deviation of lymphocyte data, a reference mean median angle light scatter of monocyte data, a reference median angle light scatter standard deviation of monocyte data, a reference mean median angle light scatter of eosinophil data and a reference median angle light scatter standard deviation of eosinophil data.

Further, the reference upper median angle light scatter data subset can include a reference mean upper median angle light scatter of neutrophil data, a reference upper median angle light scatter standard deviation of neutrophil data, a reference mean upper median angle light scatter of lymphocyte data, a reference upper median angle light scatter standard deviation of lymphocyte data, a reference mean upper median angle light scatter of monocyte data, a reference upper median angle light scatter standard deviation of monocyte data, a reference mean upper median angle light scatter of eosinophil data and a reference upper median angle light scatter standard deviation of eosinophil data.

Further, the reference lower median angle light scatter data subset can include a reference mean lower median angle light scatter of neutrophil data, a reference lower median angle light scatter standard deviation of neutrophil data, a reference mean lower median angle light scatter of lymphocyte data, a reference lower median angle light scatter standard deviation of lymphocyte data, a reference mean lower median angle light scatter of monocyte data, a reference lower median angle light scatter standard deviation of monocyte data, a reference mean lower median angle light scatter of eosinophil data and a reference lower median angle light scatter standard deviation of eosinophil data.

Further, the reference low angle light scatter data subset can include a reference mean low angle light scatter of neutrophil data, a reference low angle light scatter standard deviation of neutrophil data, a reference mean low angle light scatter of lymphocyte data, a reference low angle light scatter standard deviation of lymphocyte data, a reference mean low angle light scatter of monocyte data, a reference low angle light scatter standard deviation of monocyte data, a reference mean low angle light scatter of eosinophil data and a reference low angle light scatter standard deviation of eosinophil data.

Further, the reference axial light loss data subset can include a reference mean axial light loss of neutrophil data, a reference axial light loss standard deviation of neutrophil data, a reference mean axial light loss of lymphocyte data, a reference axial light loss standard deviation of lymphocyte data, a reference mean axial light loss of monocyte data, a reference axial light loss standard deviation of monocyte data, a reference mean axial light loss of eosinophil data and a reference axial light loss standard deviation of eosinophil data.

Further, the reference complete blood counting data can include a reference white blood cell counting data, a reference red blood cell counting data, a reference platelet counting data, a reference hemoglobin data, a reference hematocrit data, a reference platelet distribution width data, a reference monocyte distribution width data, a reference mean volume of red blood cell data, a reference mean amount of corpuscular hemoglobin data, a reference mean corpuscular hemoglobin concentration data, a reference neutrophil-to-lymphocyte ratio data and a reference platelet-to-lymphocyte ratio data. Further, the reference complete blood counting data can further include a reference nucleated red blood cell counting data.

Further, the reference white blood cell differential counting data can include a reference lymphocyte percentage data, a reference lymphocyte counting data, a reference monocyte percentage data, a reference monocyte counting data, a reference segmented neutrophil percentage data, a reference segmented neutrophil counting data, a reference band neutrophil percentage data, a reference absolute neutrophil counting data, a reference eosinophil percentage data, a reference eosinophil counting data, a reference basophil percentage data and a reference basophil counting data.

In Step 120, a model establishing step is performed, wherein the plurality of reference cell population data, the plurality of reference complete blood counting data and the plurality of reference white blood cell differential counting data are trained to achieve a convergence by a machine learning algorithm model so as to obtain a bacteremia assessing classifier. In detail, in the method 100 for assessing bacteremia, the machine learning algorithm model can be a CatBoost algorithm model, an XGBoost algorithm model, a LightGBM algorithm model, a random forest algorithm model or a logistic regression algorithm model, but the present disclosure is not limited thereto.

In Step 130, a blood analysis data of a subject is provided, wherein the blood analysis data includes a cell population data, a complete blood counting data and a white blood cell differential counting data. In detail, the subject can be an inpatient, an outpatient or an emergency case, and the subject can also be an infectious disease patient or a non-infectious disease patient, but the present disclosure is not limited thereto.

The cell population data can include a parameter data set, wherein the parameter data set can be obtained by analyzing at least one white blood cell of a blood sample of the subject by the volume, conductivity and scatter method, and the at least one white blood cell can be a neutrophil, a lymphocyte, a monocyte or an eosinophil. In detail, the parameter data set can include a cell volume data subset, a conductivity data subset, a median angle light scatter data subset, an upper median angle light scatter data subset, a lower median angle light scatter data subset, a low angle light scatter data subset and an axial light loss data subset, wherein each of the cell volume data subset, the conductivity data subset, the median angle light scatter data subset, the upper median angle light scatter data subset, the lower median angle light scatter data subset, the low angle light scatter data subset and the axial light loss data subset can include a mean data and a standard deviation data.

In detail, the cell volume data subset can include a mean volume of neutrophil data, a volume standard deviation of neutrophil data, a mean volume of lymphocyte data, a volume standard deviation of lymphocyte data, a mean volume of monocyte data, a volume standard deviation of monocyte data, a mean volume of eosinophil data and a volume standard deviation of eosinophil data.

Further, the conductivity data subset can include a mean conductivity of neutrophil data, a conductivity standard deviation of neutrophil data, a mean conductivity of lymphocyte data, a conductivity standard deviation of lymphocyte data, a mean conductivity of monocyte data, a conductivity standard deviation of monocyte data, a mean conductivity of eosinophil data and a conductivity standard deviation of eosinophil data.

Further, the median angle light scatter data subset can include a mean median angle light scatter of neutrophil data, a median angle light scatter standard deviation of neutrophil data, a mean median angle light scatter of lymphocyte data, a median angle light scatter standard deviation of lymphocyte data, a mean median angle light scatter of monocyte data, a median angle light scatter standard deviation of monocyte data, a mean median angle light scatter of eosinophil data and a median angle light scatter standard deviation of eosinophil data.

Further, the upper median angle light scatter data subset can include a mean upper median angle light scatter of neutrophil data, an upper median angle light scatter standard deviation of neutrophil data, a mean upper median angle light scatter of lymphocyte data, an upper median angle light scatter standard deviation of lymphocyte data, a mean upper median angle light scatter of monocyte data, an upper median angle light scatter standard deviation of monocyte data, a mean upper median angle light scatter of eosinophil data and an upper median angle light scatter standard deviation of eosinophil data.

Further, the lower median angle light scatter data subset can include a mean lower median angle light scatter of neutrophil data, a lower median angle light scatter standard deviation of neutrophil data, a mean lower median angle light scatter of lymphocyte data, a lower median angle light scatter standard deviation of lymphocyte data, a mean lower median angle light scatter of monocyte data, a lower median angle light scatter standard deviation of monocyte data, a mean lower median angle light scatter of eosinophil data and a lower median angle light scatter standard deviation of eosinophil data.

Further, the low angle light scatter data subset can include a mean low angle light scatter of neutrophil data, a low angle light scatter standard deviation of neutrophil data, a mean low angle light scatter of lymphocyte data, a low angle light scatter standard deviation of lymphocyte data, a mean low angle light scatter of monocyte data, a low angle light scatter standard deviation of monocyte data, a mean low angle light scatter of eosinophil data and a low angle light scatter standard deviation of eosinophil data.

Further, the axial light loss data subset can include a mean axial light loss of neutrophil data, an axial light loss standard deviation of neutrophil data, a mean axial light loss of lymphocyte data, an axial light loss standard deviation of lymphocyte data, a mean axial light loss of monocyte data, an axial light loss standard deviation of monocyte data, a mean axial light loss of eosinophil data and an axial light loss standard deviation of eosinophil data.

Further, the complete blood counting data can include a white blood cell counting data, a red blood cell counting data, a platelet counting data, a hemoglobin data, a hematocrit data, a platelet distribution width data, a monocyte distribution width data, a mean volume of red blood cell data, a mean amount of corpuscular hemoglobin data, a mean corpuscular hemoglobin concentration data, a neutrophil-to-lymphocyte ratio data and a platelet-to-lymphocyte ratio data. Further, the complete blood counting data can further include a nucleated red blood cell counting data.

Further, the white blood cell differential counting data can include a lymphocyte percentage data, a lymphocyte counting data, a monocyte percentage data, a monocyte counting data, a segmented neutrophil percentage data, a segmented neutrophil counting data, a band neutrophil percentage data, an absolute neutrophil counting data, an eosinophil percentage data, an eosinophil counting data, a basophil percentage data and a basophil counting data.

In Step 140, an assessing step is performed, wherein the blood analysis data is analyzed by the bacteremia assessing classifier so as to obtain an assessing result of bacteremia of the subject. The assessing result of bacteremia of the subject is for assessing whether the subject has the bacteremia or not so as to rapidly and accurately formulate an appropriate treatment strategy.

Therefore, by analyzing the blood analysis data of the subject by the bacteremia assessing classifier, and the bacteremia assessing classifier is established by training the reference cell population data, the reference complete blood counting data and the reference white blood cell differential counting data, the method 100 for assessing bacteremia of the present disclosure can rapidly and accurately output the assessing result of bacteremia of the subject. Hence, it is favorable for designing the subsequent medical plans of the subject, and the method 100 for assessing bacteremia of the present disclosure has excellent clinical application potential.

### [Bacteremia Assessing System of the Present Disclosure]

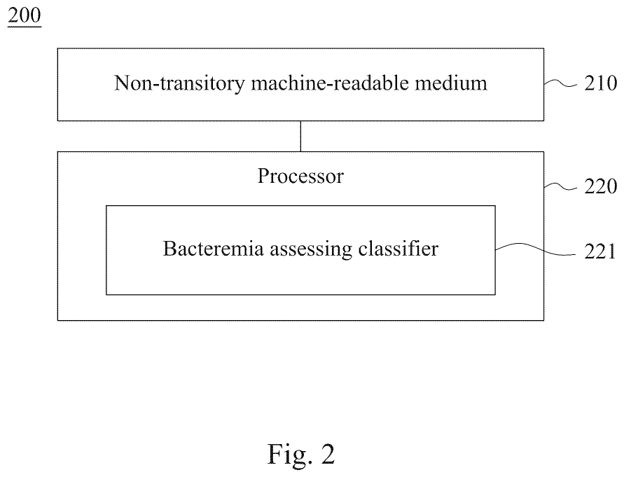

Reference is made to FIG. 2, which is a block diagram of a bacteremia assessing system 200 according to another embodiment of the present disclosure. The bacteremia assessing system 200 includes a non-transitory machine-readable medium 210 and a processor 220.

The non-transitory machine-readable medium 210 is for storing a blood analysis data of a subject, wherein the blood analysis data includes a cell population data, a complete blood counting data and a white blood cell differential counting data. Further, the details of the cell population data, the complete blood count data and the white blood cell differential count data are described in the method 100 for assessing bacteremia, so that the details thereof will not be described herein again.

Further, the non-transitory machine-readable medium 210 can be further for storing a blood analysis database, wherein the blood analysis database can include a plurality of reference cell population data, a plurality of reference complete blood counting data and a plurality of reference white blood cell differential counting data, and the plurality of reference cell population data, the plurality of reference complete blood counting data and the plurality of reference white blood cell differential counting data are used to establish a bacteremia assessing classifier. Furthermore, the details of the reference cell population data, the reference complete blood counting data and the reference white blood cell differential counting data and the establishing method of the bacteremia assessing classifier are described in the method 100 for assessing bacteremia, so that the details thereof will not be described herein again.

The processor 220 is signally connected to the non-transitory machine-readable medium 210, wherein the processor 220 includes a bacteremia assessing classifier 221, and the blood analysis data of the subject is analyzed by the bacteremia assessing classifier 221 so as to obtain an assessing result of bacteremia of the subject. The assessing result of bacteremia of the subject is for assessing whether the subject has the bacteremia or not so as to rapidly and accurately formulate an appropriate treatment strategy.

Therefore, by analyzing the blood analysis data of the subject by the bacteremia assessing classifier 221, the bacteremia assessing system 200 of the present disclosure can rapidly and accurately output the assessing result of bacteremia of the subject for designing the subsequent medical plans of the subject, and thus the bacteremia assessing system 200 of the present disclosure has excellent clinical application potential.

### EXAMPLE

### I. Blood Analysis Database

In this experiment, the blood analysis database is collected by China Medical University Hospital, and the clinical research study is approved by Institutional Ethics Committee of China Medical University Hospital, which is numbered CMUH112-REC3-043. The blood analysis database includes the blood analysis data of the electronic medical records of 20,636 subjects in the emergency department aged 20 years old and above and collected from May 1, 2021, to Jul. 31, 2021, and Mar. 1, 2022, to Dec. 31, 2022, wherein 2,166 subjects have the blood samples with positive bacterial culture results, and 18,470 subjects have the blood samples with negative bacterial culture results. Further, the plurality of reference cell population data, the plurality of reference complete blood counting data and the plurality of reference white blood cell differential counting data in the blood analysis database are obtained by the Beckman Coulter DxH 900 hematology analyzer, and the bacterial culture results of the blood samples of the subjects are obtained by the BACTEC™ FX system.

### II. Establishing Bacteremia Assessing Classifier

Before establishing the bacteremia assessing classifier, the subjects are separated into a training dataset and a testing dataset in a ratio of 80:20. In the training dataset, the performances of different machine learning algorithm models are assessed by a 5-fold cross-validation scheme. Further, in order to prevent the output results of the machine learning algorithm models from being biased towards higher values, all continuous features are scaled before training the machine learning algorithm models. In this experiment, a Standard Scaler is used to adjust the mean of the blood analysis data to zero and the standard deviation of the blood analysis data to one. Further, because the number of the blood samples with positive bacterial culture results and the number of the blood samples with negative bacterial culture results are uneven, a synthetic minority oversampling technique (SMOTE)-edited nearest neighbor (ENN) method is used to adjust the quantitative proportion of the blood samples with positive bacterial culture results to those with negative bacterial culture results. Hence, the machine learning algorithm models can be prevented from being biased towards categories with a larger number of samples in the training dataset.

Further, the plurality of reference cell population data, the plurality of reference complete blood counting data and the plurality of reference white blood cell differential counting data in the blood analysis database are trained to achieve a convergence by a machine learning algorithm model so as to obtain the bacteremia assessing classifier of the present disclosure. In this experiment, five machine learning algorithm models are respectively used to establish the bacteremia assessing classifiers of the bacteremia assessing systems of Example 1 to Example 5, so that the prediction performances of the bacteremia assessing classifiers established by the different machine learning algorithm models are analyzed. In detail, the machine learning algorithm model of the bacteremia assessing system of Example 1 is the CatBoost algorithm model, the machine learning algorithm model of the bacteremia assessing system of Example 2 is the XGBoost algorithm model, the machine learning algorithm model of the bacteremia assessing system of Example 3 is the LightGBM algorithm model, the machine learning algorithm model of the bacteremia assessing system of Example 4 is the random forest algorithm model, and the machine learning algorithm model of the bacteremia assessing system of Example 5 is the logistic regression algorithm model. Further, a feature selection and a hyperparameter tuning are not performed on the machine learning algorithm models of the bacteremia assessing systems of Example 1 to Example 5.

Furthermore, the performance quantification of the bacteremia assessing classifiers of the bacteremia assessing systems of Example 1 to Example 5 are evaluated in terms of area under the receiver operating characteristic curve (“AUROC” hereafter), the area under the precision-recall curve (“AUPRC” hereafter), the sensitivity, the F1-score, the positive predictive value (“PPV” hereafter), the negative predictive value (“NPV” hereafter) and the specificity, and a DeLong test is used to analyze the AUROC values of the bacteremia assessing systems of Example 1 to Example 5. Moreover, in this experiment, a SHapley Additive explanations (“SHAP” hereafter) method is further applied to illustrate the output results of the bacteremia assessing systems of Example 1 and the output results of the bacteremia assessing systems of Example 3, wherein the SHAP method is performed by the SHAP python 0.41.0, and the python 3.8.10 of the Google Colab platform are used to establish the bacteremia assessing classifiers of the bacteremia assessing systems of Example 1 to Example 5.

### III. Prospective Validation and External Validation

In this experiment, the prospective validation dataset used for prospective validation is collected by China Medical University Hospital, wherein the prospective validation dataset includes the blood analysis data of the electronic medical records of 3,143 subjects in the emergency department aged 20 years old and above and collected from Feb. 15, 2023, to Apr. 15, 2023, and the blood analysis data in the prospective validation dataset is obtained by the Beckman Coulter DxH 900 hematology analyzer, wherein 300 subjects have the blood samples with positive bacterial culture results, and 2,843 subjects have the blood samples with negative bacterial culture results.

Further, in this experiment, the first external validation dataset used for external validation is collected by Wei Gong Memorial Hospital, and the second external validation dataset is collected by Tainan Municipal An-Nan Hospital. In particular, the first external validation dataset includes the blood analysis data of the electronic medical records of 664 subjects in the emergency department aged 20 years old and above and collected from Dec. 1, 2022, to Jan. 31, 2023, wherein 69 subjects have the blood samples with positive bacterial culture results, and 595 subjects have the blood samples with negative bacterial culture results. The second external validation dataset includes the blood analysis data of the electronic medical records of 1,622 subjects in the emergency department aged 20 years old and above and collected from Oct. 1, 2022, to Jan. 31, 2023, wherein 118 subjects have the blood samples with positive bacterial culture results, and 1,504 subjects the have blood samples with negative bacterial culture results. Furthermore, each of the blood analysis data of the first external validation dataset and the blood analysis data of the second external validation dataset includes a cell population data, a complete blood counting data and a white blood cell differential counting data.

Moreover, in this experiment, if there is missing values in the blood analysis data of the first external validation dataset or the blood analysis data of the second external validation dataset, a data preprocessing is further performed on the first external validation dataset or the second external validation dataset. The data preprocessing is to calculate the median values from the original data of the blood analysis data in the training dataset so as to fill in the missing values. For example, in the blood analysis data of the first external validation dataset or the blood analysis data of the second external validation dataset, the missing values in the cell population data or the missing values in the complete blood counting data are replaced with the median values calculated from the cell population data in the training dataset or the complete blood counting data in the training dataset, and the missing values in the white blood cell differential counting data are replaced with zero values.

### IV. Result

### 1. Features of the Subjects

Reference is made to Table 1, which shows the analysis results of the demographics, the complete blood counting data and the white blood cell differential counting data of the subjects in the testing dataset, the prospective validation dataset, the first external validation dataset and the second external validation dataset. In detail, in Table 1, except for the values of “Number of subjects” and “Number of females”, the values of each feature are presented as medians, and the calculated interquartile range values are listed in parentheses behind the medians. Further, the value in the parentheses of “Number of females” represents the proportion of the number of female subjects in a single dataset to the number of subjects thereof.

TABLE 1

| | | | Prospective | | First external | | Second external | |
|---|---|---|---|---|---|---|---|---|
| | | | validation | | validation | | validation | |
| | Testing dataset | | dataset | | dataset | | dataset | |
| Number of subjects | 20,636 | | 3,143 | | 664 | | 1,622 | |
| Age (years old) | 64.0 | (29.0) | 62.0 | (34.0) | 69.0 | (37.0) | 66.0 | (31.0) |
| Number of females | 10,305 | (50.0) | 1,528 | (48.6) | 332 | (48.5) | 805 | (49.6) |
| White blood cells | 9.3 | (6.2) | 9.5 | (6.2) | 9.4 | (7.0) | 9.4 | (6.1) |
| (10³/μL) | | | | | | | | |
| Segmented | 79.5 | (17.2) | 80.2 | (16.7) | 79.6 | (13.7) | 76.9 | (17.9) |
| neutrophils (%) | | | | | | | | |
| Monocytes (%) | 7.0 | (4.6) | 7.0 | (4.6) | 7.0 | (5.9) | 7.0 | (5.8) |
| Lymphocytes (%) | 10.5 | (12.1) | 10.1 | (11.6) | 10.0 | (10.0) | 12.5 | (13.4) |
| Basophils (%) | 0.4 | (0.4) | 0.4 | (0.4) | 0.3 | (0.5) | 0.4 | (0.3) |
| Eosinophils (%) | 0.5 | (1.3) | 0.5 | (1.3) | 0.3 | (1.0) | 0.7 | (1.4) |
| Band neutrophils (%) | 5.8 | (9.4) | 5.1 | (13.4) | 6.0 | (14.0) | N/A | |
| Hemoglobin (g/dL) | 12.3 | (3.4) | 12.5 | (3.4) | 12.4 | (3.7) | 12.5 | (3.1) |
| Red blood cells | 4.15 | (1.15) | 4.2 | (1.2) | 4.2 | (1.2) | 4.3 | (1.1) |
| (10⁶/μL) | | | | | | | | |
| Nucleated red blood | 0 | (0.1) | 1.6 | (1.5) | 0.0 | (0.1) | 0.1 | (0.1) |
| cells (%) | | | | | | | | |
| Platelets (10³/μL) | 222.0 | (124.0) | 223.0 | (119.0) | 213.0 | (121.0) | 222.0 | (115.0) |
| Neutrophil-to- | 7.5 | (10.6) | 7.9 | (10.8) | 7.9 | (9.0) | 6.2 | (8.5) |
| lymphocyte ratio | | | | | | | | |
| Platelet-to- | 20.5 | (27.3) | 21.0 | (27.9) | 20.8 | (23.0) | 17.7 | (21.7) |
| lymphocyte ratio | | | | | | | | |
| Monocyte | 21.2 | (5.7) | 21.4 | (5.7) | N/A | | 20.5 | (5.0) |
| distribution width | | | | | | | | |
| Platelet distribution | 16.8 | (0.8) | 16.8 | (0.8) | 16.9 | (0.9) | 16.7 | (0.9) |
| width (fL) | | | | | | | | |
| Mean volume of red | 88.6 | (7.7) | 88.6 | (7.3) | 87.8 | (9.7) | 88.2 | (7.3) |
| blood cell (fL) | | | | | | | | |
| Mean amount of | 30.1 | (3) | 30.2 | (2.8) | 29.9 | (4.1) | 30.1 | (2.9) |
| corpuscular | | | | | | | | |
| hemoglobin (pg) | | | | | | | | |
| Mean corpuscular | 33.8 | (1.2) | 34.0 | (1.2) | 33.9 | (1.5) | 34.0 | (1.4) |
| hemoglobin | | | | | | | | |
| concentration (g/dL) | | | | | | | | |
| Hematocrit (%) | 36.4 | (9.7) | 36.8 | (9.8) | 36.7 | (9.7) | 36.9 | (8.5) |

As shown in Table 1, the median age of the subjects in the prospective validation dataset is younger (62±34 years old) compared to the median age of the subjects in the testing dataset (64±29 years old), and both the median age of the subjects in the first external validation dataset (69±37 years old) and the median age of the subjects in the second external validation dataset (66±31 years old) are greater than those in the testing dataset. Further, approximately a half of the subjects in the testing dataset are female, and the proportions of female subjects in the testing dataset, the prospective validation dataset, the first external validation dataset and the second external validation dataset are almost the same. Furthermore, the median white blood cell count in the testing dataset is 9.3×10⁹/L, the median proportion of segmented neutrophils in the testing dataset is 79.5%, and the median white blood cell count and the median proportion of segmented neutrophils in the prospective validation dataset, the first external validation dataset and the second external validation dataset also show similar values or proportions.

Reference is made to Table 2, which shows the bacterial culture results of the blood samples of the subjects in the testing dataset, the prospective validation dataset, the first external validation dataset and the second external validation dataset. In Table 2, “One set of culture”, “Two sets of culture” and “Three sets of culture” refer to the number of subjects who underwent one set of blood samples for bacterial culture, two sets of blood samples for bacterial culture or three sets of blood samples for the bacterial culture performed within 12 hours before or after the examination of complete blood count and white blood cell differential count, and the values in parentheses represent the proportions of the number of subjects who underwent one set of blood samples for bacterial culture, two sets of blood samples for bacterial culture or three sets of blood samples to the number of subjects in a single dataset. Further, in “Number of positives” and “Number of negatives”, the values in parentheses represent the proportions of subjects with positive or negative bacterial culture results to the number of the subjects in a single dataset. Furthermore, Table 2 also shows the species of bacteria detected during the bacterial culture of the blood samples and the number of subjects with the bacteria in their blood samples, and the values in parentheses are the proportions of subjects with the bacteria to the number of subjects in a single dataset.

TABLE 2

| | | Prospective | First external | Second external |
|---|---|---|---|---|
| | | validation | validation | validation |
| | Testing dataset | dataset | dataset | dataset |
| Number of subjects | 20,636 | 3,143 | 664 | 1,622 |

Number of subjects (%)

| | | | | | | | | |
|---|---|---|---|---|---|---|---|---|
| One set of culture | 12,543 | (60.8) | 2,000 | (63.6) | 581 | (87.5) | 454 | (28.0) |
| Two sets of culture | 8,049 | (39.0) | 1,133 | (36.0) | 78 | (11.7) | 1,166 | (71.9) |
| Three sets of culture | 44 | (0.2) | 10 | (0.3) | 5 | (0.8) | 2 | (0.1) |
| Number of positives | 2,166 | (10.5) | 300 | (9.5) | 69 | (10.4) | 118 | (7.3) |
| Number of negatives | 18,470 | (89.5) | 2,843 | (90.5) | 595 | (89.6) | 1,504 | (92.7) |
| Escherichia coli | 901 | (41.6) | 120 | (40.0) | 32 | (46.4) | 56 | (47.5) |
| Klebsiella pneumoniae | 328 | (15.1) | 29 | (9.7) | 14 | (20.3) | 21 | (17.8) |
| Staphylococcus aureus | 244 | (11.3) | 48 | (16.0) | 7 | (10.1) | 8 | (6.8) |
| Pseudomonas aeruginosa | 75 | (3.5) | 9 | (3.0) | 1 | (1.4) | 1 | (0.8) |
| Proteus mirabilis | 59 | (2.7) | 10 | (3.3) | 2 | (2.9) | 4 | (3.4) |
| Salmonella enteritidis | 56 | (2.6) | 5 | (1.7) | 2 | (2.9) | 1 | (0.8) |
| Enterococcus faecium | 39 | (1.8) | 8 | (2.7) | 0 | (0.0) | 1 | (0.8) |
| Acinetobacter baumannii | 20 | (0.9) | 3 | (1.0) | 0 | (0.0) | 1 | (0.8) |

As shown in Table 2, in the testing dataset, 10.5% of the subjects have blood samples with positive bacterial culture results, and 89.5% of the subjects have blood samples with negative bacterial culture results. Further, the proportions of the subjects with positive bacterial culture results of their blood samples in the prospective validation dataset and the first external validation dataset are lower (9.5% and 10.4%, respectively) compared with the proportion of subjects with positive bacterial culture results of their blood samples in the testing dataset, and the proportion of subjects with positive bacterial culture results of their blood samples in the second external validation dataset is the lowest, which is only 7.3%. Furthermore, Escherichia coli is the most common pathogen in the testing dataset, the prospective validation dataset, the first external validation dataset and the second external validation dataset. Moreover, in the testing dataset, the prospective validation dataset, the first external validation dataset and the second external validation dataset, Klebsiella pneumoniae is the second common pathogen, followed by Staphylococcus aureus. In the prospective validation dataset, the number of subjects whose blood samples are detected with Staphylococcus aureus accounted for 16.0% of all the subjects whose blood samples have positive bacterial culture results, and the numbers of subjects whose blood samples are detected with Klebsiella pneumoniae and Proteus mirabilis accounted for 9.7% and 3.3% of all the subjects whose blood samples have positive bacterial culture results, respectively.

### 2. Analysis of the Effectiveness of the Bacteremia Assessing System of the Present Disclosure Used to Assess the Bacteremia

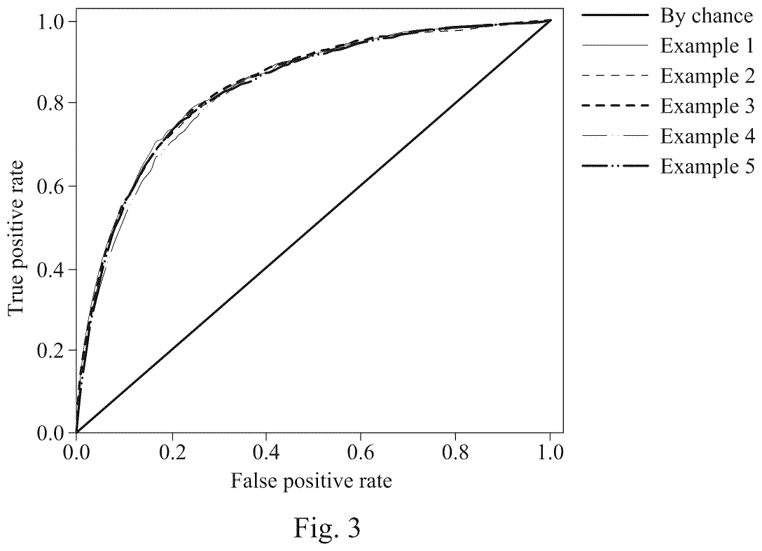

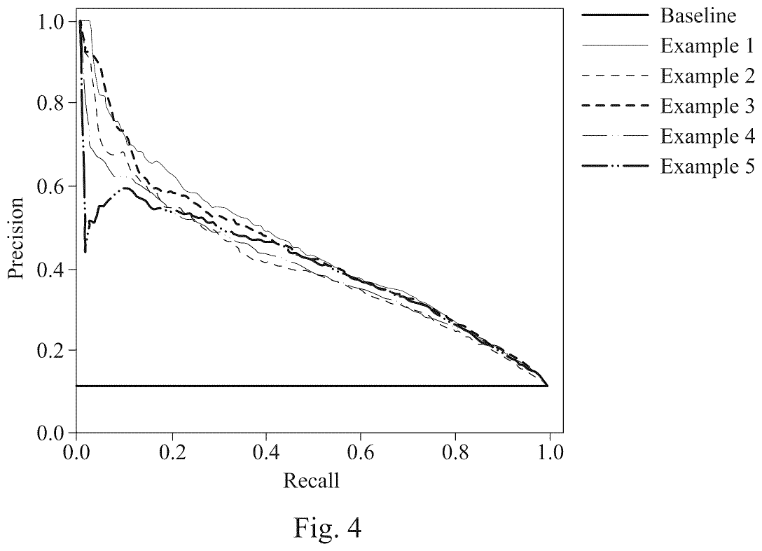

Reference is made to FIG. 3, FIG. 4 and Table 3. FIG. 3 shows the receiver operating characteristic curves of bacteremia assessing systems of Example 1 to Example 5. FIG. 4 shows the precision-recall curves of the bacteremia assessing systems of Example 1 to Example 5. Table 3 shows the analysis results of the bacteremia assessing systems of Example 1 to Example 5 used to assess the bacteremia based on the testing dataset.

TABLE 3

| | AUROC | AUPRC | F1-score | Sensitivity | Specificity | PPV | NPV |
|---|---|---|---|---|---|---|---|
| Example 1 | 0.844 ± 0.002 | 0.447 ± 0.003 | 0.445 | 0.715 | 0.826 | 0.323 | 0.962 |
| Example 2 | 0.839 ± 0.003 | 0.437 ± 0.010 | 0.439 | 0.696 | 0.829 | 0.321 | 0.959 |
| Example 3 | 0.842 ± 0.001 | 0.435 ± 0.008 | 0.435 | 0.710 | 0.820 | 0.313 | 0.961 |
| Example 4 | 0.834 ± 0.004 | 0.389 ± 0.011 | 0.391 | 0.776 | 0.746 | 0.262 | 0.966 |
| Example 5 | 0.838 ± 0.003 | 0.391 ± 0.006 | 0.323 | 0.882 | 0.586 | 0.198 | 0.977 |

As shown in FIG. 3, FIG. 4 and Table 3, all of the AUROCs of the bacteremia assessing systems of Example 1 to Example 5 are greater than 0.8, and the AUPRCs of the bacteremia assessing systems of Example 1 to Example 5 are between 0.38 and 0.45. The results show that the bacteremia assessing systems of Example 1 to Example 5 have excellent performance used to assess the bacteremia.

As further shown in Table 3, in the testing dataset, the AUROC of the bacteremia assessing system of Example 1 is 0.844±0.002, the AUPRC of the bacteremia assessing system of Example 1 is 0.447±0.003, the F1-score of the bacteremia assessing system of Example 1 is 0.445, the specificity of the bacteremia assessing system of Example 1 is 0.826, and the NPV of the bacteremia assessing system of Example 1 is 0.962. The results show that the effect of the bacteremia assessing system used to assess the bacteremia is excellent when the machine learning algorithm model of the bacteremia assessing system of the present disclosure is the CatBoost algorithm model.

Reference is made to Table 4, which shows the analysis results of the bacteremia assessing system of Example 1 and the bacteremia assessing system of Example 3 used to assess bacteremia based on the testing dataset, the prospective validation dataset, the first external validation dataset and the second external validation dataset.

TABLE 4

| | Example 1 | | Example 3 | |
|---|---|---|---|---|
| | AUROC | AUPRC | AUROC | AUPRC |
| Testing dataset | 0.844 | 0.447 | 0.842 | 0.435 |
| Prospective | 0.812 | 0.419 | 0.820 | 0.409 |
| validation dataset | | | | |
| First external | 0.844 | 0.363 | 0.837 | 0.367 |
| validation dataset | | | | |
| Second external | 0.847 | 0.426 | 0.857 | 0.437 |
| validation dataset | | | | |

As shown in Table 4, in the results of the analysis using the prospective validation dataset, the AUROC and the AUPRC of the bacteremia assessing system of Example 1 are respectively 0.812 and 0.419, and the AUROC and the AUPRC of the bacteremia assessing system of Example 3 are respectively 0.820 and 0.409. Further, in the results of the analysis using the first external validation dataset, the AUROC and the AUPRC of the bacteremia assessing system of Example 1 are respectively 0.844 and 0.363, and the AUROC and the AUPRC of the bacteremia assessing system of Example 3 are respectively 0.837 and 0.367. Furthermore, in the results of the analysis using the second external validation dataset, the AUROC and the AUPRC of the bacteremia assessing system of Example 1 are respectively 0.847 and 0.426, and the AUROC and the AUPRC of the bacteremia assessing system of Example 3 are respectively 0.857 and 0.437. According to the above, the bacteremia assessing systems of Example 1 and Example 3 both show excellent effects in assessing bacteremia.

### 3. Importance Analysis of Features

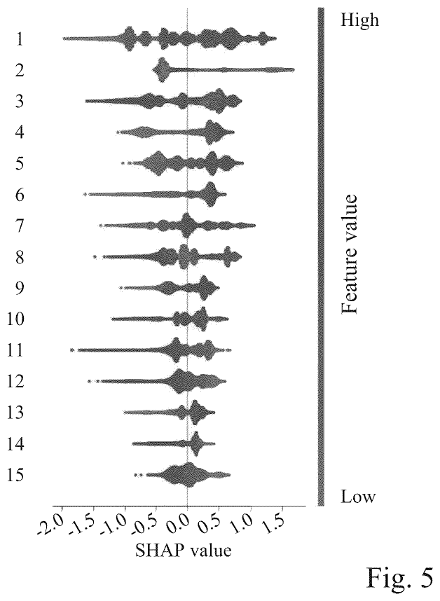

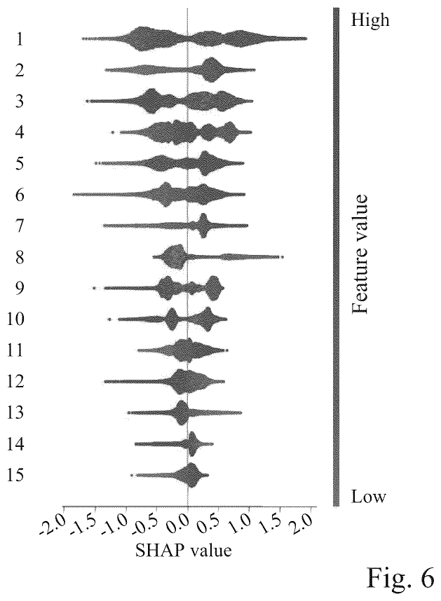

Reference is made to FIG. 5 and FIG. 6. FIG. 5 shows the analysis results of the correlations of different feature marks to the bacteremia assessing system of Example 1 used to assess bacteremia. FIG. 6 shows the analysis results of the correlations of different feature marks to the bacteremia assessing system of Example 3 used to assess bacteremia. In FIG. 5 and FIG. 6, the X-axes represent the SHAP values, which are used to assess the influence of a feature on the output of a model, and the Y-axes represent all the features used to assess the relevance of training data to developing the bacteremia. Further, the Y-axes in FIG. 5 and FIG. 6 only show the top fifteen most important features, and the order from top to bottom represents the order of importance of features. Furthermore, the names of the features corresponding to the numbers of Y-axes in FIG. 5 and FIG. 6 are shown in Table 5 and Table 6, respectively.

TABLE 5

| Number | Name | Number | Name |
|---|---|---|---|
| 1 | Mean conductivity | 2 | Percentage of |
| | of lymphocyte | | nucleated red |
| | | | blood cell |
| 3 | Mean conductivity | 4 | Neutrophil-to- |
| | of monocyte | | lymphocyte ratio |
| 5 | Volume standard | 6 | Volume standard |
| | deviation of | | deviation of |
| | monocyte | | neutrophil |
| 7 | Mean conductivity | 8 | Percentage of |
| | of eosinophil | | basophil |
| 9 | Percentage of | 10 | Mean |
| | segmented | | conductivity of |
| | neutrophil | | neutrophil |
| 11 | Platelet count | 12 | Percentage of |
| | | | monocyte |
| 13 | Mean volume | 14 | Red blood |
| | of monocyte | | cell count |
| 15 | Low angle light | | |
| | scatter standard | | |
| | deviation of | | |
| | monocyte | | |

TABLE 6

| Number | Name | Number | Name |
|---|---|---|---|
| 1 | Mean conductivity | 2 | Neutrophil-to- |
| | of lymphocyte | | lymphocyte ratio |
| 3 | Mean conductivity | 4 | Mean conductivity |
| | of monocyte | | of eosinophil |
| 5 | Percentage of | 6 | Volume standard |
| | segmented | | deviation of |
| | neutrophil | | monocyte |
| 7 | Volume standard | 8 | Percentage of |
| | deviation of | | nucleated red |
| | neutrophil | | blood cell |
| 9 | Percentage of | 10 | Mean conductivity |
| | basophil | | of neutrophil |
| 11 | Mean volume | 12 | Platelet count |
| | of neutrophil | | |
| 13 | Percentage of | 14 | Red blood |
| | monocyte | | cell count |
| 15 | Mean volume | | |
| | of monocyte | | |

In detail, in the FIG. 5 and FIG. 6, each point is one feature value for a prediction of a feature, wherein the red color represents the higher value of the feature, and the blue color represents the lower value of the feature. Further, the longer the length of the point distribution, the greater the impact of the feature on the prediction, and the shorter the length of the point distribution, the smaller the impact of the feature on the prediction. Furthermore, a positive feature value means a positive impact on the prediction, and a negative feature value means a negative impact on the prediction.

As shown in FIG. 5 and FIG. 6, in the bacteremia assessing system of Example 1, the top five most important features are the mean conductivity of lymphocyte, the percentage of nucleated red blood cell, the mean conductivity of monocyte, the mean conductivity of monocyte, the neutrophil-to-lymphocyte ratio and the volume standard deviation of monocyte. Further, in the bacteremia assessing system of Example 3, the top five most important features are the mean conductivity of lymphocyte, the neutrophil-to-lymphocyte ratio, the mean conductivity of monocyte, the mean conductivity of eosinophil and the percentage of segmented neutrophil. Furthermore, the parameter data set of the cell population data accounts for more than a half of the top ten important features of the bacteremia assessing system of Example 1 and the bacteremia assessing system of Example 3.

Further, as shown in FIG. 5, the points with high feature values of the mean conductivity of lymphocyte, the neutrophil-to-lymphocyte ratio and the volume standard deviation of monocyte are falling in the area with positive SHAP values, and it is shown that the mean conductivity of lymphocyte, the neutrophil-to-lymphocyte ratio and the volume standard deviation of monocyte are positively correlated with the bacteremia. The points with low feature values in the percentage of nucleated red blood cell and the mean conductivity of monocyte are falling in the area with positive SHAP values, and it is shown that the percentage of nucleated red blood cell and the mean conductivity of monocyte are negatively correlated with the bacteremia.

4. Analysis of the predictive effect of the bacteremia assessing classifiers trained by different types of training data

In order to analyze the impact of different types of training data on the predictive effect of the bacteremia assessing classifiers, different types of training data are trained to achieve a convergence by the CatBoost algorithm model so as to establish the bacteremia assessing system of Comparative example 1 and the bacteremia assessing system of Comparative example 2, and then the bacteremia assessing system of Comparative example 1 and the bacteremia assessing system of Comparative example 2 are compared to the bacteremia assessing system of Example 1. In detail, the training data of the bacteremia assessing system of Comparative example 1 is the cell population data, and the training data of the bacteremia assessing system of Comparative example 2 is the complete blood counting data and the white blood cell differential counting data.

Reference is made to Table 7, which shows the analysis results of the accuracy of the bacteremia assessing system of Example 1, the bacteremia assessing system of Comparative example 1 and the bacteremia assessing system of Comparative example 2 in assessing bacteremia.

TABLE 7

| Bacteremia assessing | AUROC | | |
|---|---|---|---|
| classifier | (standard deviation) | p value | |
| Example 1 | 0.844 (0.002) | Reference | — |
| Comparative example 1 | 0.836 (0.002) | 0.15 | Reference |
| Comparative example 2 | 0.810 (0.003) | <0.001 | <0.001 |

As shown in Table 7, the bacteremia assessing system of Comparative example 1 and the bacteremia assessing system of Comparative example 2 have lower AUROC compared with the bacteremia assessing system of Example 1, and it is shown that the bacteremia assessing system of Example 1 has excellent effect in assessing bacteremia.

As shown in the aforementioned results, by analyzing the blood analysis data of the subjects by the bacteremia assessing classifier, and the blood analysis data includes the cell population data, the complete blood counting data and the white blood cell differential counting data, the method for assessing bacteremia and the bacteremia assessing system of the present disclosure can rapidly and accurately output the assessing result of bacteremia of the subject. Hence, it is favorable for designing the subsequent medical plans of the subject. Further, by the bacteremia assessing classifier is established by training the reference cell population data, the reference complete blood counting data and the reference white blood cell differential counting data, the performance of the bacteremia assessing classifier used to assess the bacteremia can be effectively enhanced. Therefore, the method for assessing bacteremia and the bacteremia assessing system of the present disclosure have excellent clinical application potential.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A method for assessing bacteremia, comprising:
providing a blood analysis database, wherein the blood analysis database comprises a plurality of reference cell population data, a plurality of reference complete blood counting data and a plurality of reference white blood cell differential counting data;
performing a model establishing step, wherein the plurality of reference cell population data, the plurality of reference complete blood counting data and the plurality of reference white blood cell differential counting data are trained to achieve a convergence by a machine learning algorithm model so as to obtain a bacteremia assessing classifier;
providing a blood analysis data of a subject, wherein the blood analysis data comprises a cell population data, a complete blood counting data and a white blood cell differential counting data; and
performing an assessing step, wherein the blood analysis data is analyzed by the bacteremia assessing classifier so as to obtain an assessing result of bacteremia of the subject.

2. The method of claim 1, wherein the machine learning algorithm model is a CatBoost algorithm model, an XGBoost algorithm model, a LightGBM algorithm model, a random forest algorithm model or a logistic regression algorithm model.

3. The method of claim 1, wherein the cell population data comprises:
a parameter data set comprising a cell volume data subset, a conductivity data subset, a median angle light scatter data subset, an upper median angle light scatter data subset, a lower median angle light scatter data subset, a low angle light scatter data subset and an axial light loss data subset.

4. The method of claim 3, wherein each of the cell volume data subset, the conductivity data subset, the median angle light scatter data subset, the upper median angle light scatter data subset, the lower median angle light scatter data subset, the low angle light scatter data subset and the axial light loss data subset comprises a mean data and a standard deviation data.

5. The method of claim 4, wherein:
the cell volume data subset comprises a mean volume of neutrophil data, a mean volume of monocyte data, a volume standard deviation of neutrophil data and a volume standard deviation of monocyte data, and the conductivity data subset comprises a mean conductivity of lymphocyte data and a mean conductivity of monocyte data;
the complete blood counting data comprises a neutrophil-to-lymphocyte ratio data; and
the white blood cell differential counting data comprises a segmented neutrophil percentage data and a monocyte percentage data.

6. The method of claim 3, wherein the parameter data set is obtained by analyzing at least one white blood cell of a blood sample of the subject by a volume, conductivity and scatter (VCS) method.

7. The method of claim 6, wherein the at least one white blood cell is a neutrophil, a lymphocyte, a monocyte or an eosinophil.

8. The method of claim 1, wherein the complete blood counting data comprises a white blood cell counting data, a red blood cell counting data, a platelet counting data, a hemoglobin data, a hematocrit data, a platelet distribution width data, a monocyte distribution width data, a mean volume of red blood cell data, a mean amount of corpuscular hemoglobin data, a mean corpuscular hemoglobin concentration data, a neutrophil-to-lymphocyte ratio data and a platelet-to-lymphocyte ratio data.

9. The method of claim 1, wherein the white blood cell differential counting data comprises a lymphocyte percentage data, a lymphocyte counting data, a monocyte percentage data, a monocyte counting data, a segmented neutrophil percentage data, a segmented neutrophil counting data, a band neutrophil percentage data, an absolute neutrophil counting data, an eosinophil percentage data, an eosinophil counting data, a basophil percentage data and a basophil counting data.

10. A bacteremia assessing system, comprising:
a non-transitory machine-readable medium for storing a blood analysis data of a subject, wherein the blood analysis data comprises a cell population data, a complete blood counting data and a white blood cell differential counting data; and
a processor signally connected to the non-transitory machine-readable medium, wherein the processor comprises a bacteremia assessing classifier, and the blood analysis data is analyzed by the bacteremia assessing classifier so as to obtain an assessing result of bacteremia of the subject.

11. The bacteremia assessing system of claim 10, wherein the cell population data comprises:
a parameter data set comprising a cell volume data subset, a conductivity data subset, a median angle light scatter data subset, an upper median angle light scatter data subset, a lower median angle light scatter data subset, a low angle light scatter data subset and an axial light loss data subset.

12. The bacteremia assessing system of claim 11, wherein each of the cell volume data subset, the conductivity data subset, the median angle light scatter data subset, the upper median angle light scatter data subset, the lower median angle light scatter data subset, the low angle light scatter data subset and the axial light loss data subset comprises a mean data and a standard deviation data.

13. The bacteremia assessing system of claim 12, wherein:
the cell volume data subset comprises a mean volume of neutrophil data, a mean volume of monocyte data, a volume standard deviation of neutrophil data and a volume standard deviation of monocyte data, and the conductivity data subset comprises a mean conductivity of lymphocyte data and a mean conductivity of monocyte data;
the complete blood counting data comprises a neutrophil-to-lymphocyte ratio data; and
the white blood cell differential counting data comprises a segmented neutrophil percentage data and a monocyte percentage data.

14. The bacteremia assessing system of claim 11, wherein the parameter data set is obtained by analyzing at least one white blood cell of a blood sample of the subject by a volume, conductivity and scatter method.

15. The bacteremia assessing system of claim 14, wherein the at least one white blood cell is a neutrophil, a lymphocyte, a monocyte or an eosinophil.

16. The bacteremia assessing system of claim 10, wherein the complete blood counting data comprises a white blood cell counting data, a red blood cell counting data, a platelet counting data, a hemoglobin data, a hematocrit data, a platelet distribution width data, a monocyte distribution width data, a mean volume of red blood cell data, a mean amount of corpuscular hemoglobin data, a mean corpuscular hemoglobin concentration data, a neutrophil-to-lymphocyte ratio data and a platelet-to-lymphocyte ratio data.

17. The bacteremia assessing system of claim 10, wherein the white blood cell differential counting data comprises a lymphocyte percentage data, a lymphocyte counting data, a monocyte percentage data, a monocyte counting data, a segmented neutrophil percentage data, a segmented neutrophil counting data, a band neutrophil percentage data, an absolute neutrophil counting data, an eosinophil percentage data, an eosinophil counting data, a basophil percentage data and a basophil counting data.

18. The bacteremia assessing system of claim 10, wherein the non-transitory machine-readable medium is further for storing a blood analysis database, and the blood analysis database comprises a plurality of reference cell population data, a plurality of reference complete blood counting data and a plurality of reference white blood cell differential counting data.

19. The bacteremia assessing system of claim 18, wherein the bacteremia assessing classifier is obtained by training the plurality of reference cell population data, the plurality of reference complete blood counting data and the plurality of reference white blood cell differential counting data to achieve a convergence by a machine learning algorithm model.

20. The bacteremia assessing system of claim 19, wherein the machine learning algorithm model is a CatBoost algorithm model, an XGBoost algorithm model, a LightGBM algorithm model, a random forest algorithm model or a logistic regression algorithm model.
